(19) United States
(12) Patent Application Publication
(10) Pub. No.: US 2020/0090282 A1
(43) Pub. Date: Mar. 19, 2020

(54) METHODS FOR AUTOMATICALLY DETERMINING INJURY TREATMENT RELATION TO A MOTOR VEHICLE ACCIDENT AND DEVICES THEREOF

(71) Applicant: Mitchell International, Inc., San Diego, CA (US)

(72) Inventors: Norman E. Tyrrell, San Diego, CA (US); Olaf Wied, San Diego, CA (US); Jonathan Navarrete, San Diego, CA (US); Christopher Williamson, Escondido, CA (US); Paul Zaino, Carlsbad, CA (US)

(21) Appl. No.: 16/570,758

(22) Filed: Sep. 13, 2019

Related U.S. Application Data
(60) Provisional application No. 62/731,524, filed on Sep. 14, 2018.

(51) Int. Cl. G06Q 40/08; G16H 70/20; G06F 9/451

(52) U.S. Cl. CPC G06Q 40/08; G06F 9/451; G16H 70/20

(57) ABSTRACT

Methods, non-transitory computer readable media, and insurance claim analysis devices are disclosed that generate an injury severity score based on a delta velocity value for a damaged motor vehicle and occupant data for an occupant of the damaged motor vehicle or motor vehicle data associated with the damaged motor vehicle. A first set of condition indications are identified based on a correlation of the injury severity score with a stored mapping of condition indications to injury severity scores. A determination is made when one or more of the first set of condition indications correspond to one or more of a second set of condition indications in injury data for an electronic insurance claim. The electronic insurance claim is automatically adjudicated based on a likelihood value generated based on the determination. The likelihood value is indicative of whether a reported injury of the occupant resulted from an associated motor vehicle accident.

This application claims the benefit of U.S. Provisional Patent Application Ser. No. 62/731,524, filed on Sep. 14, 2018, which is hereby incorporated by reference in its entirety.

### FIELD

This technology generally relates to methods, non-transitory computer readable media, and devices for automated data and image analysis to determine injury treatment relation to a motor vehicle accident.

### BACKGROUND

Adjusters, including auto injury adjusters, are faced with the challenge of efficiently and reliably assessing the likely causality and relation of reported or treated injuries to the facts of loss in an accident, such as a motor vehicle accident, for example. Manual adjuster determinations regarding whether a particular medical treatment should be considered for payment are currently subjective, inconsistent, susceptible to inaccuracies, and not scalable.

Additionally, there is currently no automated or systematic way to analyze physical damage evidence (e.g., motor vehicle damage images) to inform the injury analysis and whether certain injuries should be excluded from claim adjudication consideration. Further, injury determinations made from physical damage repair estimates are inaccurate, and occur too late in the insurance claim lifecycle. Accordingly, injury analysis currently has a negative impact on the efficiency of the end-to-end insurance claim adjudication process.

### SUMMARY

A method for automatically determining injury treatment relation to a motor vehicle accident is disclosed that includes generating, by an insurance claim analysis device, an injury severity score. The injury severity score is generated based on a delta velocity value for a damaged motor vehicle involved in a motor vehicle accident and at least one of occupant data for an occupant of the damaged motor vehicle or motor vehicle data associated with the damaged motor vehicle. A first set of condition indications are identified based on a correlation of the injury severity score with a stored mapping of condition indications to injury severity scores. A determination is made when one or more of the first set of condition indications correspond to one or more of a second set of condition indications in injury data for an electronic insurance claim. The electronic insurance claim is automatically adjudicated based on a likelihood value generated based on the determination. The likelihood value is indicative of whether a reported injury of the occupant resulted from an associated motor vehicle accident.

An insurance claim analysis device is disclosed that includes memory including programmed instructions stored thereon and one or more processors configured to execute the stored programmed instructions to generate an injury severity score. The injury severity score is generated based on a delta velocity value for a damaged motor vehicle involved in a motor vehicle accident and at least one of occupant data for an occupant of the damaged motor vehicle or motor vehicle data associated with the damaged motor vehicle. A first set of condition indications are identified based on a correlation of the injury severity score with a stored mapping of condition indications to injury severity scores. A determination is made when one or more of the first set of condition indications correspond to one or more of a second set of condition indications in injury data for an electronic insurance claim. The electronic insurance claim is automatically adjudicated based on a likelihood value generated based on the determination. The likelihood value is indicative of whether a reported injury of the occupant resulted from an associated motor vehicle accident.

A non-transitory machine readable medium is disclosed that has stored thereon instructions for automatically determining injury treatment relation to a motor vehicle accident including executable code that, when executed by one or more processors, causes the processors to generate an injury severity score. The injury severity score is generated based on a delta velocity value for a damaged motor vehicle involved in a motor vehicle accident and at least one of occupant data for an occupant of the damaged motor vehicle or motor vehicle data associated with the damaged motor vehicle. A first set of condition indications are identified based on a correlation of the injury severity score with a stored mapping of condition indications to injury severity scores. A determination is made when one or more of the first set of condition indications correspond to one or more of a second set of condition indications in injury data for an electronic insurance claim. The electronic insurance claim is automatically adjudicated based on a likelihood value generated based on the determination. The likelihood value is indicative of whether a reported injury of the occupant resulted from an associated motor vehicle accident.

This technology has a number of associated advantages including providing methods, non-transitory computer readable media, and insurance claim analysis devices that facilitate improved accuracy, consistency, and efficiency with respect to analyzing images and data associated with insurance claims to automatically recommend inclusion or exclusion of associated reported injuries from claim adjudication consideration. This technology advantageously utilizes machine learning models to automatically analyze damaged motor vehicle images and other insurance claim data in order to generate and utilize delta velocity values and injury severity scores. The injury severity scores are advantageously mapped to condition indications in order to facilitate an improved, automated determination regarding whether an injury reported as part of an insurance claim likely resulted from an associated motor vehicle accident.

### BRIEF DESCRIPTION OF THE DRAWINGS

FIG. 1 a block diagram of a network environment with an exemplary insurance claim analysis device;

FIG. 2 is a block diagram of the exemplary insurance claim analysis device of FIG. 1;

FIG. 3 is a flowchart of an exemplary method for automatically determining injury treatment relation to a motor vehicle accident;

FIG. 4 is an exemplary mapping of condition indications to injury severity scores; and

FIG. 5 is a screenshot of an exemplary graphical user interface (GUI) that can be used to report injury treatment relation to a motor vehicle accident.

### DETAILED DESCRIPTION

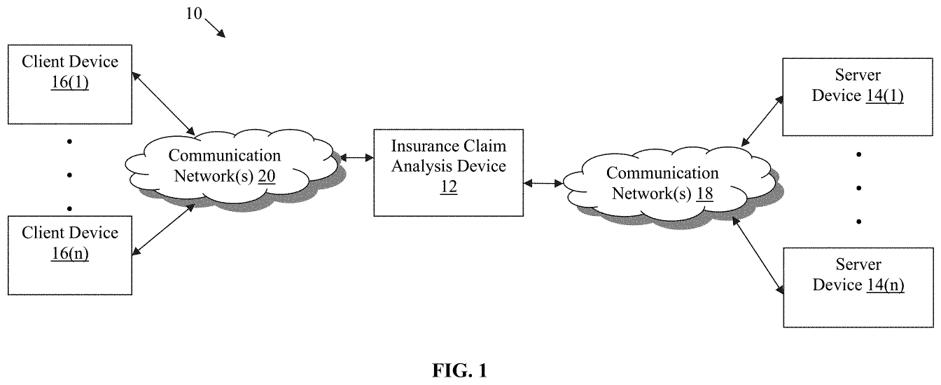

Referring to FIG. 1, an exemplary network environment 10 with an exemplary insurance claim analysis device 12 is illustrated. The insurance claim analysis device 12 in this example is coupled to a plurality of server devices 14(1)-14(n) and a plurality of client devices 16(1)-16(n) via communication network(s) 18 and 20, respectively, although the insurance claim analysis device 12, server devices 14(1)-14(n), and/or client devices 16(1)-16(n), may be coupled together via other topologies. Additionally, the network environment 10 may include other network devices such as one or more routers and/or switches, for example, which are well known in the art and thus will not be described herein. This technology provides a number of advantages including methods, non-transitory computer readable media, and insurance claim analysis devices that use machine learning models, an automated analysis of image(s) of the damaged motor vehicle, and determination of a delta velocity value and injury severity score for the practical application of determining a likelihood that a reported injury of an occupant of a motor vehicle resulted from an accident involving the motor vehicle during the automated processing of insurance claims.

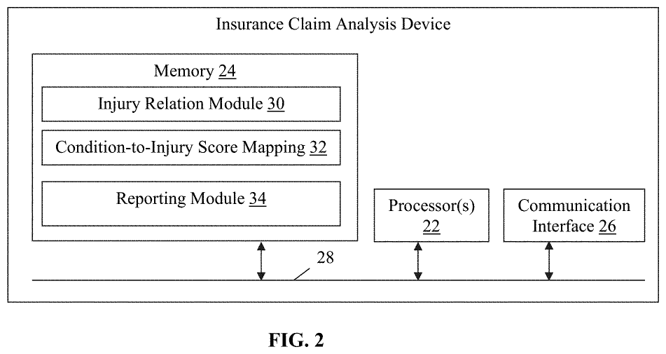

Referring to FIGS. 1-2, the insurance claim analysis device 12 in this example includes processor(s) 22, a memory 24, and/or a communication interface 26, which are coupled together by a bus 28 or other communication link, although the insurance claim analysis device can include other types and/or numbers of elements in other configurations. The processor(s) 22 of the insurance claim analysis device 12 may execute programmed instructions stored in the memory 24 for the any number of the functions described and illustrated herein. The processor(s) 22 may include one or more CPUs or general purpose processors with one or more processing cores, for example, although other types of processor(s) can also be used.

The memory 24 of the insurance claim analysis device 12 stores these programmed instructions for one or more aspects of the present technology as described and illustrated herein, although some or all of the programmed instructions could be stored elsewhere. A variety of different types of memory storage devices, such as random access memory (RAM), read only memory (ROM), hard disk, solid state drives, flash memory, or other computer readable medium which is read from and written to by a magnetic, optical, or other reading and writing system that is coupled to the processor(s) 22, can be used for the memory 24.

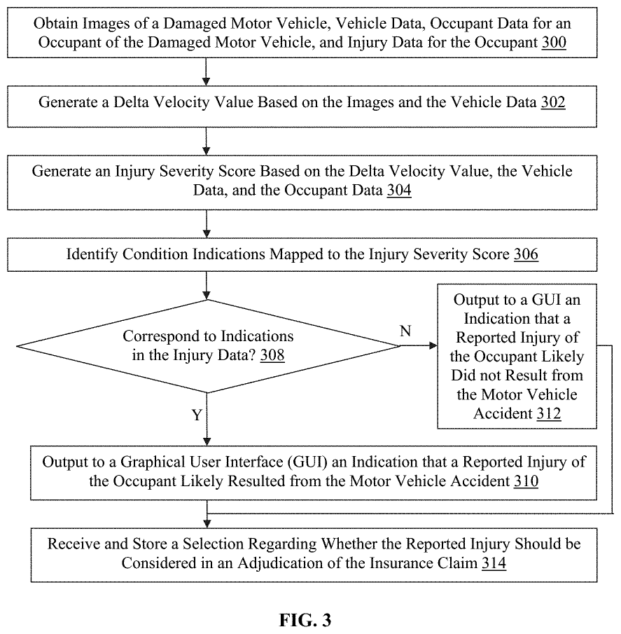

Accordingly, the memory 24 can store application(s) that can include executable instructions that, when executed by the insurance claim analysis device 12, cause the insurance claim analysis device 12 to perform actions, such as to transmit, receive, or otherwise process network messages, for example, and to perform other actions described and illustrated below with reference to FIGS. 3-5. The application(s) can be implemented as modules or components of other application(s). Further, the application(s) can be implemented as operating system extensions, module, plugins, or the like.

Even further, the application(s) may be operative in a cloud-based computing environment. The application(s) can be executed within or as virtual machine(s) or virtual server(s) that may be managed in a cloud-based computing environment. Also, the application(s), and even the insurance claim analysis device 12 itself, may be located in virtual server(s) running in a cloud-based computing environment rather than being tied to one or more specific physical network computing devices. Also, the application(s) may be running in one or more virtual machines (VMs) executing on the insurance claim analysis device 12. Additionally, in one or more embodiments of this technology, virtual machine(s) running on the insurance claim analysis device 12 may be managed or supervised by a hypervisor.

In this particular example, the memory 24 includes an injury relation module 30, a condition-to-injury score mapping 32, and a reporting module 34, although the memory 24 can include other policies, modules, databases, or applications, for example. The injury relation module 30 in this example is configured to ingest images of a damaged motor vehicle, occupant data, and injury data. Based on the ingested images and vehicle data, the injury relation module 30 is configured to apply a first machine learning model to automatically determine a delta velocity value associated with an accident involving the damaged motor vehicle. The injury relation module 30 is further configured to apply a second machine learning model to generate an injury severity score based on the delta velocity value, the vehicle data, and the occupant data.

With the resulting injury severity score, the injury relation module 30 in this example utilizes the condition-to-injury score mapping 32 to identify condition indications, and determines whether the condition indications correspond with condition indications in the ingested injury data. In one example, the condition-to-injury score mapping 32 includes a mapping of condition indications in the form of International Statistical Classification of Diseases and Related Health Problems (ICD) codes to injury scores in the form of Abbreviated Injury Scale (AIS) scores, although other types of condition indication and/or injury severity scores can also be used in other examples.

The injury data can be reported as part of, or extracted from, an electronic insurance claim. Accordingly, the injury relation module 30 can automatically determine, from images of a damaged motor vehicle, a likelihood that reported injuries of an occupant of the damaged motor vehicle resulted from the motor vehicle accident that is associated with an insurance claim in which the injuries were reported. The operation of the injury relation module 30 is described and illustrated in more detail later with reference to FIG. 3.

The reporting module 34 in this example is configured to output at least an indication of the likelihood generated by the injury relation module 30 to the client devices 12(1)-12(n). In one example, the reporting module 34 can generate a graphical user interface (GUI) that includes the indication of the likelihood. In another example, the indication of the likelihood can be provided to a third party or end user GUI or device in response a call received via a provided application programming interface (API), for example. Accordingly, the likelihood can be output by the claim analysis device 12 via a provided GUI or via API consumption, and the likelihood can also be provided via other manners in other examples.

The reporting module 34 in this particular example is further configured to store a selection received from the client devices 12(1)-12(n) regarding whether a reported injury should be considered in an adjudication process associated with an insurance claim. Accordingly, the output likelihood in this example can inform the decision by an insurance adjuster, for example, as to whether a reported injury should be considered or was actually a result of a motor vehicle accident associated with an insurance claim.

The communication interface 26 of the insurance claim analysis device 12 operatively couples and communicates between the insurance claim analysis device 12, the server devices 14(1)-14(n), and/or the client devices 16(1)-16(n), which are all coupled together by the communication network(s) 16 and 18, although other types and/or numbers of communication networks or systems with other types and/or numbers of connections and/or configurations to other devices and/or elements can also be used.

By way of example only, the communication network(s) 16 and 18 can include local area network(s) (LAN(s)) or wide area network(s) (WAN(s)), and can use TCP/IP over Ethernet and industry-standard protocols, although other types and/or numbers of protocols and/or communication networks can be used. The communication network(s) 16 and 18 in this example can employ any suitable interface mechanisms and network communication technologies including, for example, teletraffic in any suitable form (e.g., voice, modem, and the like), Public Switched Telephone Network (PSTNs), Ethernet-based Packet Data Networks (PDNs), combinations thereof, and the like.

The insurance claim analysis device 12 can be a standalone device or integrated with one or more other devices or apparatuses, such as one or more of the server devices 14(1)-14(n), for example. In one particular example, the insurance claim analysis device 12 can include or be hosted by one of the server devices 14(1)-14(n), and other arrangements are also possible.

Each of the server devices 14(1)-14(n) in this example includes processor(s), a memory, and a communication interface, which are coupled together by a bus or other communication link, although other numbers and/or types of network devices could be used. The server devices 14(1)-14(n) in this example host content associated with insurance carrier(s) including insurance claim data that can include images of damaged motor vehicle, vehicle data, occupant data, and/or injury data, for example.

Although the server devices 14(1)-14(n) are illustrated as single devices, one or more actions of the server devices 14(1)-14(n) may be distributed across one or more distinct network computing devices that together comprise one or more of the server devices 14(1)-14(n). Moreover, the server devices 14(1)-14(n) are not limited to a particular configuration. Thus, the server devices 14(1)-14(n) may contain a plurality of network devices that operate using a master/slave approach, whereby one of the network devices of the server devices 14(1)-14(n) operate to manage and/or otherwise coordinate operations of the other network devices.

The server devices 14(1)-14(n) may operate as a plurality of network devices within a cluster architecture, a peer-to peer architecture, virtual machines, or within a cloud architecture, for example. Thus, the technology disclosed herein is not to be construed as being limited to a single environment and other configurations and architectures are also envisaged.

The client devices 16(1)-16(n) in this example include any type of computing device that can interface with the insurance claim analysis device to submit data and/or receive GUI(s). Each of the client devices 16(1)-16(n) in this example includes a processor, a memory, and a communication interface, which are coupled together by a bus or other communication link, although other numbers and/or types of network devices could be used.

The client devices 16(1)-16(n) may run interface applications, such as standard web browsers or standalone client applications, which may provide an interface to communicate with the insurance claim analysis device 12 via the communication network(s) 20. The client devices 16(1)-16(n) may further include a display device, such as a display screen or touchscreen, and/or an input device, such as a keyboard, for example. In one example, the client devices 16(1)-16(n) can be utilized by insurance adjusters to facilitate an improved analysis of insurance claims as described and illustrated herein, although other types of client devices 16(1)-16(n) utilized by other types of users can also be used in other examples.

Although the exemplary network environment 10 with the insurance claim analysis device 12, server devices 14(1)-14(n), client devices 16(1)-16(n), and communication network(s) 16 and 18 are described and illustrated herein, other types and/or numbers of systems, devices, components, and/or elements in other topologies can be used. It is to be understood that the systems of the examples described herein are for exemplary purposes, as many variations of the specific hardware and software used to implement the examples are possible, as will be appreciated by those skilled in the relevant art(s).

One or more of the devices depicted in the network environment 10, such as the insurance claim analysis device 12, client devices 16(1)-16(n), or server devices 14(1)-14(n), for example, may be configured to operate as virtual instances on the same physical machine. In other words, one or more of the insurance claim analysis device 12, client devices 16(1)-16(n), or server devices 14(1)-14(n) may operate on the same physical device rather than as separate devices communicating through communication network(s) 16 and 18. Additionally, there may be more or fewer insurance claim analysis devices, client devices, or server devices than illustrated in FIG. 1.

In addition, two or more computing systems or devices can be substituted for any one of the systems or devices in any example. Accordingly, principles and advantages of distributed processing, such as redundancy and replication also can be implemented, as desired, to increase the robustness and performance of the devices and systems of the examples. The examples may also be implemented on computer system(s) that extend across any suitable network using any suitable interface mechanisms and traffic technologies, including by way of example only wireless networks, cellular networks, PDNs, the Internet, intranets, and combinations thereof.

The examples may also be embodied as one or more non-transitory computer readable media, such as the memory 24, having instructions stored thereon for one or more aspects of the present technology as described and illustrated by way of the examples herein. The instructions in some examples include executable code that, when executed by one or more processors, such as the processor(s) 22, cause the processors to carry out steps necessary to implement the methods of the examples of this technology that are described and illustrated herein.

An exemplary method of automatically determining injury treatment relation to a motor vehicle accident will now be described with reference to FIGS. 3-5. Referring more specifically to FIG. 3, a flowchart of an exemplary method of automatically determining injury treatment relation to a motor vehicle accident is illustrated. In step 300 in this example, the insurance claim analysis device 12 obtains images of a damaged motor vehicle, vehicle data, occupant data for an occupant of the damaged motor vehicle, and injury data for the occupant. The ingested images and data can be obtained from one or more of the server devices 14(1)-14(n) and/or one of the client devices 16(1)-16(n), for example, and can be associated with an insurance claim associated with an accident involving the damaged motor vehicle that was submitted to an insurance carrier. Accordingly, the occupant can be a claimant in some examples.

The vehicle data can include a type of the damaged motor vehicle, an age of the damaged motor vehicle, a size of the damaged motor vehicle, a weight of the damaged motor vehicle, an area of impact on the damaged motor vehicle, a damage extent, one or more crush measurements, or whether the damaged motor vehicle was drivable subsequent to the motor vehicle accident, for example, although other types of vehicle data can be used in other examples. In some examples, the occupant data includes demographic data regarding the occupant, such as an occupant age, weight, height, or gender, where the occupant was sitting in the damaged motor vehicle, a point of impact on the damaged motor vehicle, or whether an airbag deployed as a result of the associated motor vehicle accident, for example, although other types of occupant data can also be used in other examples. The injury data can include condition indication(s) (e.g., ICD code(s)) associated with an injury or treatment reported as part of an insurance claim associated with the motor vehicle accident, for example.

In step 302, the insurance claim analysis device 12 generates a delta velocity value for the damaged motor vehicle involved in the motor vehicle accident associated with the insurance claim. In order to generate the delta velocity value, the insurance claim analysis device 12 automatically analyzes the obtained images of the damaged motor vehicle and applies a machine learning model based on the analysis and at least a portion of the obtained vehicle data. In one example, the delta velocity can be generated as described and illustrated in more detail in U.S. Provisional Patent Application Ser. No. 62/731,259, filed on Sep. 14, 2018, and entitled “Methods for Improved Delta Velocity Prediction Using Machine Learning and Devices Thereof,” which is incorporated herein by reference in its entirety, although other methods of generating the delta velocity value can also be used in other examples.

In step 304, the insurance claim analysis device 12 applies a second machine learning model to generate an injury severity score (e.g., an AIS score) based on the delta velocity value, at least a portion of the vehicle data, and at least a portion of the occupant data. The insurance claim analysis device 12 can utilize data regarding where the occupant was sitting in the damaged motor vehicle, occupant demographic data, the area of impact on the damaged motor vehicle, and whether the car was drivable, among other factors and data, for example, to generate the injury severity score.

The machine learning model can optionally be trained using data obtained from the National Automotive Sampling System (NASS) hosted by the National Highway Traffic Safety Administration (NHTSA), and can optionally be updated based on manual feedback or implicit learning, for example, although other methods for training and/or maintaining the second machine learning model can also be used in other examples. It is not well-understood, routine or convention activity in the art to correlate the delta velocity value to an injury severity score via the application of a machine learning model, which improves the accuracy and efficiency of the overall insurance claim processing with respect to the relationship of the reported injury treatments.

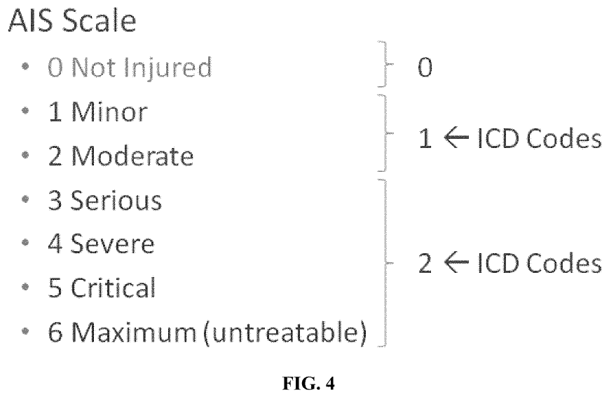

In step 306, the insurance claim analysis device 12 identifies a set of condition indications based on the stored condition-to-injury score mapping 32. Referring more specifically to FIG. 4, an exemplary mapping of condition indications to injury severity scores is illustrated. In this example, the condition-to-injury score mapping 30 includes AIS scores mapped to ICD codes that correspond with medical treatments, although other types of condition indications or injury severity scores can also be used in other examples.

The AIS scores of 1 and 2 in this example are mapped to a set of ICD codes and the AIS scores of 3-6 are mapped to another set of ICD codes, although any number of AIS scores could be mapped to any number of ICD codes in other examples. Utilizing the stored condition-to-injury score mapping 30 to identify particular condition indications that correlate with a particular injury severity score provides a practical application of facilitating more effective and automated determinations regarding the relation of an injury treatment to a motor vehicle accident, and is not well-understood, routine, or conventional in the art.

Referring back to FIG. 3, in step 308, the insurance claim analysis device 12 determines whether the condition indication(s) in the injury data obtained in step 300 match condition indication(s) in the set of condition indications identified in step 306. The conditions indication(s) in the injury data can correspond with medical treatments of the occupant of the damaged motor vehicle that were reported on an associated insurance claim, for example.

Accordingly, the insurance claim analysis device 12 compares the condition indication(s) in the injury data to the identified set of condition indications that correspond with a generated injury severity score to determine whether the condition indication(s) are associated with a reported injury that likely resulted from the motor vehicle accident. If the insurance claim analysis device 12 determines in step 308 that the condition indication in the injury data matches a condition indication in the set of condition indications identified in step 306, then the Yes branch is taken to step 310.

In step 310, the insurance claim analysis device 12 generates a GUI that includes a likelihood value indicative of whether a reported injury of the occupant resulted from the motor vehicle accident associated with the insurance claim. The GUI can be output to a requesting one of the client devices 16(1)-16(n) to allow an adjuster user, for example, to obtain an automated indication regarding whether the reported injury is likely a result of the motor vehicle accident and should be considered in an adjudication of the insurance claim. Referring back to step 308, if the insurance claim analysis device 12 determines that the condition indication in the injury data does not match a condition indication in the set of condition indications identified in step 306, then the No branch is taken to step 312.

In step 312, the insurance claim analysis device 12 optionally generates a GUI that includes an indication that the reported injury of the occupant does not likely result from the motor vehicle accident associated with the insurance claim. In other examples, the likelihood value and/or indication that the reported injury of the occupant does not likely result from the motor vehicle accident associated with the insurance claim can be provided for API consumption by an end user of one of the client devices 16(1-16(n). Subsequent to outputting the GUI in step 310 or 312, the insurance claim analysis device 12 proceeds to step 314.

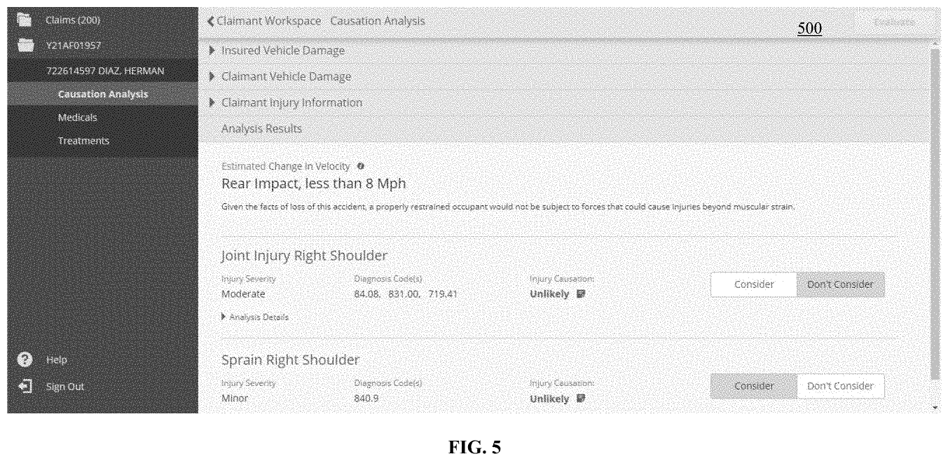

In step 314, the insurance claim analysis device 12 receives and stores a selection regarding whether the reported injury should be considered in an adjudication of the insurance claim. Referring more specifically to FIG. 5, a screenshot of an exemplary GUI 500 is illustrated. In this example, the GUI 500 includes a portion of the data obtained as described earlier with reference to step 300 of FIG. 3. In particular, the GUI 500 includes injury data such as an injury severity score or an equivalent thereof (e.g., “minor” or “moderate”) and associated condition indications, which in this example are ICD codes referred to as “diagnosis code(s)” for an injury treatment reported in an insurance claim associated with a motor vehicle accident.

The GUI 500 further includes an indication regarding whether the reported injuries likely resulted from the associated motor vehicle accident. In particular, the “joint injury right shoulder” and “sprain right shoulder” reported injuries are indicated as unlikely to have been caused by the motor vehicle accident associated with the insurance claim. The indications could have been output on the GUI 500 as described in detail earlier with reference to step 310 of FIG. 3, for example. Additionally, the GUI 500 in this example includes “Consider” and “Don't Consider” buttons that are configured to receive, and facilitate storage of, a selection regarding whether the associated reported injury should be considered in an adjudication of the insurance claim. Other types of GUIs with other types of information and/or methods of outputting the indications and/or receiving or storing the selections could also be used in other examples.

With this technology, a determination regarding whether an injury reported as part of an insurance claim likely resulted from an associated motor vehicle accident can advantageously be determined based on an automated analysis of insurance claim data, including damaged motor vehicle images. This technology utilizes machine learning models to facilitate improved accuracy, consistency, and efficiency with respect to analyzing images and data associated with insurance claims to automatically recommend inclusion or exclusion of associated reported injuries from claim adjudication consideration. The automated generation and utilization of delta velocity values and injury severity scores mapped to condition indications of this technology is not well-understood, routine, or conventional in the art and facilitates an end-to-end, practical, automated, and improved analysis of insurance claim data.

Having thus described the basic concept of the invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and scope of the invention. Additionally, the recited order of processing elements or sequences, or the use of numbers, letters, or other designations therefore, is not intended to limit the claimed processes to any order except as may be specified in the claims. Accordingly, the invention is limited only by the following claims and equivalents thereto.

What is claimed is:

1. A method for automatically determining injury treatment relation to a motor vehicle accident, the method comprising:
generating, by an insurance claim analysis device, an injury severity score based on a delta velocity value for a damaged motor vehicle involved in a motor vehicle accident and at least one of occupant data for an occupant of the damaged motor vehicle or motor vehicle data associated with the damaged motor vehicle;
identifying, by the insurance claim analysis device, a first set of one or more condition indications based on a correlation of the injury severity score with a stored mapping of condition indications to injury severity scores;
determining, by the insurance claim analysis device, when one or more of the first set of condition indications correspond to one or more of a second set of condition indications in injury data for an electronic insurance claim associated with the motor vehicle accident; and
automatically adjudicating, by the insurance claim analysis device, the electronic insurance claim based on a likelihood value generated based on the determination, the likelihood value indicative of whether a reported injury of the occupant resulted from the motor vehicle accident.

2. The method of claim 1, wherein the condition indications comprise International Statistical Classification of Diseases and Related Health Problems (ICD) codes and the injury severity score comprises an Abbreviated Injury Scale (AIS) score.

3. The method of claim 1, further comprising applying, by the insurance claim analysis device, one or more machine learning models to automatically generate the injury severity score or automatically analyze obtained images of the damaged motor vehicle to generate the delta velocity value.

4. The method of claim 1, further comprising:
outputting, by the insurance claim analysis device, the likelihood value via a graphical user interface (GUI); and
receiving, by the insurance claim analysis device and via the GUI, a selection regarding whether the reported injury should be considered in the adjudication of the insurance claim.

5. The method of claim 1, wherein the vehicle data comprises one or more of a type of the motor vehicle, an age of the motor vehicle, a size of the motor vehicle, a weight of the motor vehicle, an area of impact on the motor vehicle, a damage extent, one or more crush measurements, or whether the motor vehicle was drivable subsequent to the motor vehicle accident.

6. The method of claim 1, wherein the occupant data comprises one or more of demographic data regarding the occupant comprising one or more of an occupant age, weight, height, or gender, where the occupant was sitting in the motor vehicle, a point of impact on the motor vehicle, or whether an airbag deployed as a result of the motor vehicle accident.

7. An insurance claim analysis device, comprising memory comprising programmed instructions stored thereon and one or more processors configured to execute the stored programmed instructions to:
generate an injury severity score based on an obtained delta velocity value for a damaged motor vehicle involved in a motor vehicle accident and at least one of occupant data for an occupant of the damaged motor vehicle or motor vehicle data associated with the damaged motor vehicle;
identify a first set of one or more condition indications based on a correlation of the injury severity score with a stored mapping of condition indications to injury severity scores;
determine when one or more of the first set of condition indications correspond to one or more of a second set of condition indications in injury data for an electronic insurance claim associated with the motor vehicle accident; and
automatically adjudicate the electronic insurance claim based on a likelihood value generated based on the determination, the likelihood value indicative of whether a reported injury of the occupant resulted from the motor vehicle accident.

8. The insurance claim analysis device of claim 7, wherein the condition indications comprise International Statistical Classification of Diseases and Related Health Problems (ICD) codes and the injury severity score comprises an Abbreviated Injury Scale (AIS) score.

9. The insurance claim analysis device of claim 7, wherein the processors are further configured to execute the stored programmed instructions to apply one or more machine learning models to automatically generate the injury severity score or automatically analyze obtained images of the damaged motor vehicle to generate the delta velocity value.

10. The insurance claim analysis device of claim 7, wherein the processors are further configured to execute the stored programmed instructions to:
output the likelihood value via a graphical user interface (GUI); and
receive, via the GUI, a selection regarding whether the reported injury should be considered in the adjudication of the insurance claim.

11. The insurance claim analysis device of claim 7, wherein the vehicle data comprises one or more of a type of the motor vehicle, an age of the motor vehicle, a size of the motor vehicle, a weight of the motor vehicle, an area of impact on the motor vehicle, a damage extent, one or more crush measurements, or whether the motor vehicle was drivable subsequent to the motor vehicle accident.

12. The insurance claim analysis device of claim 7, wherein the occupant data comprises one or more of demographic data regarding the occupant comprising one or more of an occupant age, weight, height, or gender, where the occupant was sitting in the motor vehicle, a point of impact on the motor vehicle, or whether an airbag deployed as a result of the motor vehicle accident.

13. A non-transitory machine readable medium having stored thereon instructions for automatically determining injury treatment relation to a motor vehicle accident comprising executable code that, when executed by one or more processors, causes the processors to:
generate an injury severity score based on an obtained delta velocity value for a damaged motor vehicle involved in a motor vehicle accident and at least one of occupant data for an occupant of the damaged motor vehicle or motor vehicle data associated with the damaged motor vehicle;
identify a first set of one or more condition indications based on a correlation of the injury severity score with a stored mapping of condition indications to injury severity scores;
determine when one or more of the first set of condition indications correspond to one or more of a second set of condition indications in injury data for an electronic insurance claim associated with the motor vehicle accident; and
automatically adjudicate the electronic insurance claim based on a likelihood value generated based on the determination, the likelihood value indicative of whether a reported injury of the occupant resulted from the motor vehicle accident.

14. The non-transitory machine readable medium of claim 13, wherein the condition indications comprise International Statistical Classification of Diseases and Related Health Problems (ICD) codes and the injury severity score comprises an Abbreviated Injury Scale (AIS) score.

15. The non-transitory machine readable medium of claim 13, wherein the executable code, when executed by the processors, further causes the processors to apply one or more machine learning models to automatically generate the injury severity score or automatically analyze obtained images of the damaged motor vehicle to generate the delta velocity value.

16. The non-transitory machine readable medium of claim 13, wherein the executable code, when executed by the processors, further causes the processors to:
output the likelihood value via a graphical user interface (GUI); and
receive, via the GUI, a selection regarding whether the reported injury should be considered in the adjudication of the insurance claim.

17. The non-transitory machine readable medium of claim 13, wherein the vehicle data comprises one or more of a type of the motor vehicle, an age of the motor vehicle, a size of the motor vehicle, a weight of the motor vehicle, an area of impact on the motor vehicle, a damage extent, one or more crush measurements, or whether the motor vehicle was drivable subsequent to the motor vehicle accident.

18. The non-transitory machine readable medium of claim 13, wherein the occupant data comprises one or more of demographic data regarding the occupant comprising one or more of an occupant age, weight, height, or gender, where the occupant was sitting in the motor vehicle, a point of impact on the motor vehicle, or whether an airbag deployed as a result of the motor vehicle accident.
